(19) United States
(12) Patent Application Publication
(10) Pub. No.: US 2025/0109393 A1
(43) Pub. Date: Apr. 3, 2025

(54) METHOD FOR SEPARATING HMW DNA AND/OR UHMW DNA FROM A CARRIER MEMBER, TO WHICH THE DNA IS RELEASABLY ATTACHED

(71) Applicant: HAMILTON BONADUZ AG, Bonaduz (CH)

(72) Inventors: Birgit Ottenwälder, Bonaduz (CH); Jannik Donner, Bonaduz (CH); Kim Jana Neufeld, Bonaduz (CH); Enrique Bayo, Bonaduz (CH); Nadine Thaler, Bonaduz (CH); Andreas Maissen, Bonaduz (CH); Gian Jörimann, Bonaduz (CH); Lucas Durandi, Bonaduz (CH)

(21) Appl. No.: 18/727,931

(22) PCT Filed: Jan. 13, 2023
(86) PCT No.: PCT/EP2023/050694

§ 371 Date: Jul. 10, 2024

(30) Foreign Application Priority Data
Jan. 19, 2022 (DE) 10 2022 101 145.3

(51) Int. Cl. C12N 15/10; B01L 3/00

(52) U.S. Cl. CPC C12N 15/1006; B01L 3/5021; B01L 3/5025; B01L 3/50855; B01L 2200/025; B01L 2200/026; B01L 2300/0609

(57) ABSTRACT

The invention relates to a method for separating HMW DNA and/or UHMW DNA from a carrier member (8), to which the DNA is releasably attached, using a centrifugation container set (2) having at least one centrifugation container (10a, 10b, 10c, 10d, 10e, 10f, 10g, 10h) and at least one collection vessel (40a, 40b, 40c, 40d, 40e, 40f, 40g, 40h), wherein the centrifugation container (10a, 10b, 10c, 10d, 10e, 10f, 10g, 10h) is separably plugged on the collection vessel (40a, 40b, 40c, 40d, 40e, 40f, 40g, 40h), wherein the centrifugation container (10a, 10b, 10c, 10d, 10e, 10f, 10g, 10h) has a container wall (14), which surrounds a container interior (12), and a container base (16) having at least one outflow opening (20), wherein the outflow opening (20) forms at least one flow path (24) from the container interior (12) of the centrifugation container (10a, 10b, 10c, 10d, 10e, 10f, 10g, 10h) to a vessel interior (42) of the collection vessel (40a, 40b, 40c, 40d, 40e, 40f, 40g, 40h) for a fluid to be centrifuged, wherein the at least one centrifugation container (10a, 10b, 10c, 10d, 10e, 10f, 10g, 10h) and the at least one collection vessel (40a, 40b, 40c, 40d, 40e, 40f, 40g, 40h) are geometrically coordinated with one another such that there is a free distance (a) from an outlet opening (22) of the outflow opening (20) of the centrifugation container (10a, 10b, 10c, 10d, 10e, 10f, 10g, 10h) to a vessel base (44) of the collection vessel (40a, 40b, 40c, 40d, 40e, 40f, 40g, 40h) in the direction of a main extension axis (H1) of the centrifugation container (10a, 10b, 10c, 10d, 10e, 10f, 10g, 10h), wherein the free distance (a) is selected such that possible threads of the DNA detach from the outlet opening (22).

The invention relates to a method for separating HMW DNA and/or UHMW DNA from a carrier member, to which the DNA is releasably attached. The invention further relates to a centrifugation container for plugging on a collection vessel for separating HMW DNA and/or UHMW DNA from a carrier member, to which the DNA is releasably attached. The invention further relates to a centrifugation container set for separating HMW DNA and/or UHMW DNA from a carrier member, to which the DNA is releasably attached. The invention further relates to an automatic laboratory machine. The invention further relates to a use of the centrifugation container, of the centrifugation container set, or of the automatic laboratory machine for separating HMW DNA and/or UHMW DNA from a carrier member, to which the DNA is releasably attached.

HMW DNA, or high molecular weight DNA, refers to DNA having DNA molecules with a length from at least 50 kilobases (kb) up to around 300 kb. Such DNA molecules have a high molecular weight. Among other uses, HMW DNA serves as a starting material for sequencing methods involving long reading operations or for optical genome mapping methods by which, for example, relatively large structural variants and long regions having sequence motif repeats can be identified in the genome.

UHMW DNA, or ultra-high molecular weight DNA, refers to DNA having DNA molecules with a length of at least 300 kb. Such DNA molecules have a very high molecular weight. Among other uses, UHMW DNA serves as a starting material for sequencing methods involving very long reading operations or for optical genome mapping methods by which, for example, very large structural variants and very long regions having sequence motif repeats can be identified in the genome. The analysis of UHMW DNA can uncover structural variants and genome changes that are not detectable using HMW DNA analysis owing to the shorter length of the DNA molecules of HMW DNA. By comparison with DNA molecules of a shorter length and thus of lower molecular weights, UHMW DNA is more difficult to isolate and handle since UHMW DNA above a particular concentration is highly viscous, has a sticky consistency, and pulls threads. Moreover, extremely careful handling is needed in order to preserve the integrity of the UHMW DNA molecules.

To isolate HMW DNA and/or UHMW DNA from cells or tissue, it is known to use a carrier member, for example a magnetic disk, having a surface that binds HMW DNA and/or UHMW DNA. The disk is transferred into a lysate of cells or tissue, and then the HMW DNA and/or UHMW DNA is precipitated from the lysate by the addition of isopropanol. In the process, the HMW DNA and/or UHMW DNA releasably attaches to the surface of the disk. The disk is then washed together with the DNA attached thereto.

As is known, the HMW DNA and/or UHMW DNA is separated from the disk, to which the DNA is releasably attached, in the following way: First, the disk is incubated in an elution buffer in a first reaction vessel having a capacity of, for example, 1.5 ml. Next, a first part of the elution buffer together with the DNA contained therein is transferred into a second reaction vessel; up to now this has been done manually by pipetting. In this case, the first part of the elution buffer is the part that contains DNA molecules of HMW DNA and/or UHMW DNA, which can be separated from the disk by means of pipetting. The second part of the elution buffer together with the DNA contained therein initially remains on the disk owing to the stickiness of the long DNA molecules and the adhesion of the long DNA molecules to the disk surface, and is then separated from the disk by way of a centrifuging step. Due to the cup-shaped design of the first reaction vessel, during the centrifuging the disk remains stuck a few millimeters above the base of the first reaction vessel while the second part of the elution buffer collects below the disk at the base of the first reaction vessel. Once the first reaction vessel containing the disk has been centrifuged, the centrifuged-off second part of the elution buffer together with the DNA contained therein is likewise transferred into the second reaction vessel manually by pipetting. To facilitate this pipetting step and prevent the DNA from undesirably binding to the disk again, the disk can be removed from the first reaction vessel beforehand using a magnet. In this case, the two parts of the elution buffer can also be combined in the first reaction vessel as an alternative to the second reaction vessel.

The above-described known procedure is used, for example, when using commercially available kits for isolating and purifying HMW DNA and/or UHMW DNA from various companies, for example Circulomics Inc. and Bionano Genomics Inc.

The disadvantage of the above-described known procedure for separating HMW DNA and/or UHMW DNA from the disk is that the procedure is laborious and cannot be carried out in an automated manner. In addition, at least two pipetting steps are needed, each of which brings a risk of intact HMW DNA or UHMW DNA molecules being lost, in particular owing to shear forces, which lead to loss of size of the DNA molecules. Moreover, the at least two pipetting steps each run the risk of the DNA being contaminated and the risk of samples being accidentally mixed up. Furthermore, in the case of UHMW DNA, special pipette tips having a wide pipette opening are needed for pipetting solutions containing UHMW DNA, in order to reduce shear forces.

Therefore, there is a need to show ways in which HMW DNA and/or UHMW DNA can be separated from a disk or another carrier member, to which the HMW DNA and/or UHMW DNA is releasably attached, without the aforementioned disadvantages of the known procedure.

The object of the invention is solved by a method for separating HMW DNA and/or UHMW DNA from a carrier member, to which the DNA is releasably attached, using a centrifugation container set having at least one centrifugation container and at least one collection vessel, wherein the centrifugation container is separably plugged on the collection vessel, wherein the centrifugation container has a container wall, which surrounds a container interior, and a container base having at least one outflow opening, wherein the outflow opening forms at least one flow path from the container interior of the centrifugation container to a vessel interior of the collection vessel for a fluid to be centrifuged, wherein the at least one centrifugation container and the at least one collection vessel are geometrically coordinated with one another such that there is a free distance from an outlet opening of the outflow opening of the centrifugation container to a vessel base of the collection vessel in the direction of a main extension axis of the centrifugation container, wherein the free distance is selected such that possible threads of the DNA detach from the outlet opening, comprising the steps of:
incubating the carrier member, to which the DNA is releasably attached, in an elution buffer in the container interior of the centrifugation container,
centrifuging the centrifugation container set in order to separate the elution buffer containing the DNA from the carrier member, wherein during the centrifuging the elution buffer containing the DNA flows through the outflow opening into the vessel interior of the collection vessel while the carrier member is retained in the centrifugation container at a distance from the outflow opening by a retaining device.

HMW DNA refers to DNA having DNA molecules with a length from at least 50 kilobases (kb) up to around 300 kb. Such DNA molecules have a high molecular weight. HMW DNA is short for high molecular weight DNA. Among other uses, HMW DNA serves as a starting material for sequencing methods involving long reading operations or for optical genome mapping methods by which, for example, relatively large structural variants and long regions having sequence motif repeats (repetitive regions) can be identified in bacterial, human, animal, or plant genomes and analytically resolved. In this case, relatively large structural variants refer to relatively large DNA sequence changes in the genome, for example deletions, insertions, duplications, and translocations. Such structural variants can involve a very broad size spectrum, from around 1 kb up to the megabase (Mb) range. The length of the DNA molecules of HMW DNA determines which size spectrum of structural variants and genome changes can be captured on the basis of analyses of individual molecules.

UHMW DNA refers to DNA having DNA molecules with a length of at least 300 kb. Such DNA molecules have a very high molecular weight. UHMW DNA is short for ultra-high molecular weight DNA. Among other uses, UHMW DNA serves as a starting material for sequencing methods involving very long reading operations or for optical genome mapping methods by which, for example, 10 very large structural variants and very long regions having sequence motif repeats can be identified in bacterial, human, animal, or plant genomes and analytically resolved. The analysis of UHMW DNA can uncover structural variants and genome changes that are not detectable using HMW DNA analysis owing to the shorter length of the DNA molecules of HMW DNA. By comparison with DNA molecules of a shorter length and thus of lower molecular weights, UHMW DNA is more difficult to isolate and handle since UHMW DNA above a particular concentration is highly viscous, has a sticky consistency, and pulls threads. Moreover, extremely careful handling is needed in order to preserve the integrity of the UHMW DNA molecules.

At the beginning of the method, the HMW DNA and/or UHMW DNA is releasably attached to the carrier member. The attachment to the carrier member is based on a non-covalent bond between the DNA and the carrier member, in which the DNA adheres to the surface of the carrier member. For this purpose, the carrier member has a DNA-binding (DNA-attaching) surface and shape that favors a gentle attachment of the DNA molecules of the HMW DNA and/or UHMW DNA. The attachment of the DNA to the surface of the carrier member is brought about by, among other things, electrostatic interactions and salt bridges between the DNA and the surface of the carrier member. To isolate HMW DNA and/or UHMW DNA, the carrier member is typically transferred into a lysate of cells or tissue, and then the HMW DNA and/or UHMW DNA is precipitated from the lysate by the addition of isopropanol. In the process, the DNA releasably attaches to the surface of the carrier member. Before the DNA is separated from the carrier member, typically the carrier member together with the DNA attached thereto is washed. Therefore, the method according to the invention can be part of a method for isolating HMW DNA and/or UHMW DNA from cells or tissue.

The carrier member can have numerous different shapes. All that is crucial is that, as described above, it has a surface to which the HMW DNA and/or UHMW DNA can releasably attach. By way of example, the carrier member can be a flat round or oval platelet or a flat platelet having one or more corners. In another example, the carrier member can have the shape of a ball, a C₆₀ fullerene molecule, an oblate ellipsoid of revolution, or a prolate ellipsoid of revolution.

Preferably, the carrier member is magnetic, so that it can be moved by means of a magnet.

By way of example, the carrier member can be a substantially flat round platelet. A platelet of this kind is also called a slice or disk and is supplied in various commercially available kits for isolating HMW DNA and/or UHMW DNA. The disk format particularly effectively protects the HMW DNA and/or UHMW DNA from damage. Moreover, the disk format eliminates the liquid dead volume. In this way, a particularly efficient method for isolating DNA is facilitated and high purity of the isolated DNA is achieved. In addition, the disk is simpler to handle compared with a bead or another carrier member having a highly curved surface. In particular, the disk can be transported more simply, thereby making it simpler to, among other things, introduce the carrier member into the container interior of the centrifugation container. In the case of a magnetic carrier member, the disk can be transported more simply by means of a magnet compared with a bead. A further advantage of the disk is its large surface area in relation to its volume. Moreover, most containers used in laboratories have a round cross section, so that the round shape of the disk facilitates the handling of the disk. The diameter of the disk can, for example, be in a range from 1.0 mm to 6.0 mm, preferably in a range from 3.0 mm to 5.0 mm, and particularly preferably can be approximately 4.0 mm, with diameters of 3.0 mm, 4.0 mm, or 5.0 mm being customary. According to an embodiment, the carrier member is therefore a disk (slice) having a diameter of 1.0 mm to 6.0 mm, preferably of 3.0 mm to 5.0 mm, more preferably of approximately 4.0 mm. The thickness of the disk can be in a range from 0.4 mm to 0.45 mm, for example. The disk can be a solid magnetic disk, for example. The disk can comprise a core made of a thermoplastic polymer, for example.

In another example, the carrier member can have the shape of a small ball, i.e., a bead. The bead can also be referred to as a granule or as granulate, respectively. In this case, a plurality of beads are typically used for one DNA sample. The beads can be glass beads or plastics beads, for example. The diameter of the beads can, for example, be in a range from 1.0 mm to 6.0 mm, preferably in a range from 3.0 mm to 5.0 mm or from 2.0 mm to 4.0 mm, and particularly preferably can be approximately 4.0 mm.

The surface of the carrier member can, for example, be covered with micro- and nanostructured silica (silicon dioxide), wherein the surface of the carrier member preferably is covered with the micro- and nanostructured silica to a high density. A surface of this kind has a high DNA binding capacity, that is to say, it can releasably attach large amounts of HMW DNA and/or UHMW DNA. The structuring of the surface protects the DNA against shear forces such that the DNA molecules are not damaged when the DNA attaches to the carrier member and when the DNA releases from the carrier member. The micro- and nanostructured silica can, for example, have fold structures (for example having a length of approximately 10 nm to approximately 100 μm) and/or discrete, substantially planar flakes (for example having a length of approximately 10 nm to approximately 100 μm).

The micro- and nanostructured silica can be applied to a thin flat substrate, for example a polymer substrate, as a coating having a thickness of, for example, 2 nm to 500 nm. A carrier member of this kind is also called a silica nanomembrane. Such carrier members are known for isolating DNA.

In another example, the carrier member can have a roughened surface, for example a roughened plastics surface. Carrier members having a roughened plastics surface are known for isolating DNA.

In another example, the carrier member can have a smooth surface. Carrier members having a smooth surface are known in particular for isolating HMW DNA.

Preferably, the carrier member is a disk the surface of which is covered with micro- and nanostructured silica. A carrier member of this kind is particularly well suited for isolating UHMW DNA. Preferably, therefore, the DNA is UHMW DNA when a disk the surface of which is covered with micro- and nanostructured silica is used as the carrier member. However, the disk can also releasably attach shorter DNA molecules of HMW DNA.

In another example, the carrier member can be a glass bead having a smooth surface. A carrier member of this kind is particularly well suited for isolating HMW DNA. Preferably, therefore, the DNA is HMW DNA when a glass bead having a smooth surface is used as the carrier member. However, the glass bead can also releasably attach longer DNA molecules of UHMW DNA.

The centrifugation container set comprises the at least one centrifugation container and the at least one collection vessel. The centrifugation container can be plugged on the collection vessel at the beginning of the method, or the centrifugation container is plugged on the collection vessel while the method is being carried out. The carrier member and the elution buffer can be introduced into the centrifugation container, namely into the container interior, when the centrifugation container is plugged on the collection vessel or before the centrifugation container is plugged on the collection vessel. Preferably, the carrier member and the elution buffer are introduced into the container interior separately, i.e., each one by itself, such that the contact between the carrier member and the elution buffer occurs in the container interior.

The centrifugation container is separably plugged on the collection vessel and thus separably connected to the collection vessel. In other words, the centrifugation container can be separated from the collection vessel without the centrifugation container and/or the collection vessel being damaged, in particular without them being damaged in such a way as to render them unsuitable for being used as intended. In particular, the centrifugation container is connected to the collection vessel so as to be separable in particular without the use of tools. Being connected so as to be separable without the use of tools means that the centrifugation container can be separated from the collection vessel purely by hand, without the use of any aids.

The main extension axis of the centrifugation container is the axis of the centrifugation container in its largest extension. When the centrifugation container is in a filling position, for example, said main extension axis extends from the top end of said centrifugation container to the bottom end thereof, the direction of the main extension axis coinciding with the direction of gravity in the filling position. A filling position refers to the position in which the centrifugation container finds itself when the carrier member and the elution buffer are introduced into the centrifugation container. This can be done through a filling opening located at a top end of the centrifugation container in the direction of gravity.

The centrifugation container thus has at least two openings: The filling opening for filling the container interior with the carrier member and the elution buffer, and the at least one outflow opening, which forms the at least one flow path from the container interior of the centrifugation container to the vessel interior of the collection vessel for the fluid to be centrifuged. In the method, the fluid to be centrifuged is the elution buffer together with the HMW DNA and/or UHMW DNA contained therein.

Between the outlet opening of the outflow opening of the centrifugation container and the vessel base of the collection vessel there is the free distance in the direction of the main extension axis of the centrifugation container, wherein the free distance is selected such that possible threads of the DNA detach from the outlet opening. The free distance is implemented by the centrifugation container and the collection vessel being accordingly geometrically coordinated with one another. The free distance ensures that possible threads of the DNA detach from the outlet opening while the centrifugation container set is being centrifuged. The threads can form especially in the case of UHMW DNA, since UHMW DNA, and thus the elution buffer containing UHMW DNA, has the property of pulling threads owing to its high viscosity. The threads appear particularly between the outlet opening of the outflow opening of the centrifugation container and the vessel base of the collection vessel when the centrifugation container set is centrifuged. The free distance between the outlet opening of the centrifugation container and the vessel base of the collection vessel therefore has to be sufficiently large so that such threads tear. This also ensures that possible threads of the DNA that might form between the carrier member and the vessel base of the collection vessel during the centrifuging tear. Therefore, the free distance is important especially for separating the UHMW DNA, or the elution buffer containing UHMW DNA, from the outlet opening. Such threads may also appear in the case of HMW DNA having relatively long DNA molecules, in particular DNA molecules having a length of approximately 250 kb and more. By contrast, in the case of solutions of shorter DNA molecules, in particular of DNA molecules having a length of less than 250 up to 300 kb, fewer and shorter such threads appear. In general, as the length of the DNA molecules decreases, fewer and shorter threads appear, or no threads appear at all, since the corresponding DNA-containing solutions are less viscous.

The appearance of possible threads of the DNA during the centrifuging depends on the viscosity of the DNA-containing elution buffer. Besides the length of the DNA molecules, the viscosity of the DNA-containing elution buffer also depends on the concentration of the accordingly long DNA molecules in the elution buffer. When there is a relatively low concentration of relatively long DNA molecules, in particular of DNA molecules having a minimum length of approximately 250 kb, the viscosity of the DNA-containing elution buffer is lower, that is to say, fewer threads or no threads appear by comparison with a higher concentration of said DNA molecules. The DNA concentration primarily depends on the amount of material used for isolating the DNA, for example on the quantity of cells used for this purpose. Moreover, the DNA concentration depends on the efficiency of the DNA isolation. Both factors influence the amount of DNA that is releasably attached to the carrier member. The more cells used for isolating the DNA and the greater the efficiency of the DNA isolation, the higher the concentration of the relatively long DNA molecules in the elution buffer and thus the higher the viscosity of the DNA-containing elution buffer. As the viscosity of the DNA-containing elution buffer increases, the free distance from the outlet opening of the centrifugation container to the vessel base of the collection vessel must be increased accordingly so that possible threads of the DNA detach from the outlet opening during the centrifuging. The volume of elution buffer used also influences the DNA concentration of the elution buffer and thus its viscosity. Typically, the volume of elution buffer is kept as low as possible.

As the viscosity of the DNA-containing elution buffer increases, the free distance from the outlet opening of the centrifugation container to the vessel base of the collection vessel must be increased accordingly. To establish which minimum free distance should be selected so that possible threads of the DNA reliably detach from the outlet opening during the centrifuging, tests can be carried out using centrifugation container sets having different free distances.

To separate UHMW DNA from the carrier member, the free distance is preferably at least 6.0 mm, more preferably at least 8.0 mm, more preferably at least 9.5 mm, more preferably at least 15.0 mm, more preferably at least 20.0 mm, more preferably at least 25.0 mm. The same applies to separating HMW DNA and UHMW DNA from the carrier member.

To separate HMW DNA from the carrier member, the free distance is preferably at least 2.0 mm, more preferably at least 4.0 mm, more preferably at least 6.0 mm, more preferably at least 8.0 mm, more preferably at least 9.5 mm, more preferably at least 15.0 mm, more preferably at least 20.0 mm, more preferably at least 25.0 mm.

The carrier member to which the DNA is releasably attached is first incubated in the elution buffer in the container interior of the centrifugation container. The elution buffer is a fluid by which the DNA can be separated from the carrier member during the centrifuging. By way of example, the elution buffer can be water or a slightly alkaline Tris-HCl solution. Some of the DNA molecules, namely especially the DNA molecules that are not that long, can already release from the carrier member during the incubation since shorter DNA molecules do not adhere to the surface of the carrier member as strongly. The longer DNA molecules of the HMW DNA and/or UHMW DNA, however, do not release from the carrier member until the centrifuging. Possible shorter DNA molecules released from the carrier member during the incubation are not yet separated from the carrier member before the centrifuging since the carrier member is still in the elution buffer together with the DNA molecules dissolved therein. As used herein, separating the DNA from the carrier member refers to a spatial separation of the DNA from the carrier member.

The outflow opening is formed such that the fluid to be centrifuged can flow through the outflow opening only under the effect of a centrifugal force. For this purpose, the outflow opening preferably has a diameter of 0.6 mm to 1.6 mm, more preferably from 0.6 mm to 1.0 mm. The preferred upper limit of the diameter of the outflow opening depends on the volume used and the composition of the elution buffer, and also on the length of the outflow opening in the direction of the main extension axis of the centrifugation container, that is to say, on the length of the outflow opening from the container base to the outlet opening. The preferred lower limit of the diameter of the outflow opening depends, among other things, on the viscosity of the DNA-containing elution buffer. In any case, the diameter of the outflow opening should be selected such that the DNA-containing elution buffer does not block the outflow opening during the centrifuging. While the carrier member is being incubated in the elution buffer, no centrifugal force acts on the centrifugation container. Thus, no elution buffer flows through the outflow opening during the incubation.

The length of the outflow opening from the container base to the outlet opening can be 2.0 to 3.0 mm, for example.

In the method, the entire volume of the elution buffer containing the DNA is transferred into the collection vessel in one step while the centrifugation container set is being centrifuged.

During the centrifuging, the elution buffer together with the DNA dissolved therein, and thus the DNA, is separated from the carrier member and transferred into the vessel interior of the collection vessel through the outflow opening of the centrifugation container while the carrier member is retained in the centrifugation container by the retaining device. This means that the retaining device is designed such that the fluid to be centrifuged-here the elution buffer containing the DNA—can reach the outflow opening of the centrifugation container while the carrier member cannot reach the outflow opening. By way of example, the retaining device can comprise a plurality of retaining elements, with at least one channel, in which the fluid to be centrifuged can reach the outflow opening, being formed between the plurality of retaining elements. The at least one channel can be formed such that the fluid to be centrifuged can reach the outflow opening under the effect of gravity. Alternatively, the at least one channel can be formed such that the fluid to be centrifuged can reach the outflow opening only under the effect of a centrifugal force.

The carrier member is retained at a distance from the outflow opening by the retaining device. Owing to a distance being provided between the carrier member and the outflow opening by the retaining device, it is ensured that the carrier member does not close the outflow opening of the centrifugation container in a covering or sealing manner. In other words, the retaining device interacts with the carrier member such that the carrier member does not close the outflow opening of the centrifugation container. Any such closure would prevent the elution buffer containing the DNA from flowing out of the centrifugation container into the collection vessel during the centrifuging. Without the centrifuging, the DNA cannot be fully separated from the carrier member. This is due to the strong adhesion of the longer DNA molecules of the HMW DNA and/or UHMW DNA to the surface of the carrier member.

The distance between the carrier member and the outflow opening can be in a range from 1.0 mm to 5.0 mm, for example. The distance can be 3.5 mm, for example.

After the centrifuging, the DNA is in the collection vessel ready to be analyzed or processed further.

Using the method according to the invention, the HMW DNA and/or UHMW DNA can be fully separated from the carrier member in a reliable manner. This reliably prevents the DNA from undesirably binding to the carrier member again.

The method according to the invention is less laborious and thus less time- and staff-intensive than the previously known procedure for separating HMW DNA and/or UHMW DNA from the carrier member. The method according to the invention comprises fewer steps and thus shortens the operation for separating the HMW DNA and/or UHMW DNA from the carrier member to which the DNA is releasably attached. In particular, the at least two pipetting steps required in the previously known procedure are omitted, that is to say, the difficult pipetting of the first part of the elution buffer together with the DNA contained therein into a separate reaction vessel is omitted, as is the pipetting for combining the first and second parts of the elution buffer together with the DNA contained therein. In omitting the pipetting steps, the method according to the invention reduces the risk of material loss of DNA and the risk of contamination of the DNA. In addition, the process reliability is increased since samples of the first or second part of the elution buffer cannot be accidentally mixed up. At the same time, in the case of UHMW DNA, the special pipette tips needed for pipetting UHMW DNA-containing solutions are not required.

The material loss of HMW DNA and/or UHMW DNA in manual pipetting steps is caused not only by the possibility of some of the viscous long DNA molecules getting stuck on the pipette tip, but also by the fact that DNA molecules may be damaged or torn into smaller sections by the pipette tip. Both of these are prevented by the method according to the invention.

Omitting the pipetting steps means that the DNA can be treated with particular care in the method according to the invention. The longer DNA molecules of the HMW DNA and/or UHMW DNA in particular can be gently separated from the carrier member. Using the method, therefore, it is possible to also obtain particularly long DNA molecules having a length of more than 250 kb, which, owing to their length, are particularly suitable for sequencing methods involving long reading operations or for optical genome mapping methods.

The DNA is preferably UHMW DNA or a mixture of HMW DNA and UHMW DNA. Since UHMW DNA comprises DNA molecules having a length of at least 300 kb, the above-described advantages of the method according to the invention become particularly significant in the case of UHMW DNA.

A further advantage of the method according to the invention is that it can be carried out in an automated manner. Carrying out the method in an automated manner, for example by means of pipetting robots or an automatic laboratory machine, saves on time and staff. In addition, the automation allows for a standardized method sequence and for concurrent processing of a plurality of carrier members to which HMW DNA and/or UHMW DNA is releasably attached.

According to one embodiment, a centrifugation container is used the retaining device of which has at least one retaining element that extends to a certain extent into the container interior from an internal face of the container wall and/or an internal face of the container base. This allows the at least one retaining element and the centrifugation container to be formed in one piece and/or of the same material. For example, the at least one retaining element can be molded on the internal face of the container wall and/or on the internal face of the container base. The centrifugation container having the at least one retaining element can thus be produced particularly simply in one manufacturing step without having to assemble two or more components.

According to a further embodiment, the at least one retaining element is a rib, a knob, or a bar. The retaining element can thus have a basic shape that is particularly simple to produce. The rib can, for example, be of an octahedral shape having congruent trapezoidal side faces, or have a cuboidal basic shape. The octahedral shape having the congruent trapezoidal side faces is preferable in this case since it makes it simpler to eject the centrifugation container from the injection mold when the centrifugation container is produced using injection-molding technology.

According to a further embodiment, the at least one retaining element has side faces, the side faces having an incline toward the central longitudinal axis of the centrifugation container. The resulting draft angles make it simpler to eject the centrifugation container from the injection mold when the centrifugation container is produced using injection-molding technology.

According to another embodiment, a centrifugation container is used the retaining device of which is formed by the internal face of the container wall and/or the internal face of the container base. For this purpose, the internal face of the container wall and/or the internal face of the container base can have at least one channel which is set into the container wall and/or container base outwardly and in which the fluid to be centrifuged can reach the outflow opening. In this embodiment, the retaining device does not have a retaining element.

According to another embodiment, a centrifugation container is used the retaining device of which is formed by the shape of the container interior of the centrifugation container. For this purpose, the shape of the container interior and the shape of the carrier member are geometrically coordinated with one another such that the carrier member is retained at a distance from the outflow opening. For example, when a circular carrier member is used, the container interior can have an elliptical cross section, the dimensions of which ensure that the circular carrier member is retained at a distance from the outflow opening while the fluid to be centrifuged can reach the outflow opening. The retaining device does not need a retaining element in this embodiment either.

The invention further relates to a centrifugation container for plugging on a collection vessel for separating HMW DNA and/or UHMW DNA from a carrier member, to which the DNA is releasably attached, wherein the centrifugation container has:
a container wall, which surrounds a container interior, and
a container base having at least one outflow opening, wherein the outflow opening forms at least one flow path from the container interior to an external environment of the centrifugation container for a fluid to be centrifuged, wherein the container interior is designed for incubating the carrier member, to which the DNA is releasably attached, in an elution buffer, and wherein the centrifugation container has a retaining device that is designed for retaining the carrier member at a distance from the outflow opening.

The centrifugation container is suitable in particular for separating HMW DNA and/or UHMW DNA from a carrier member, to which the DNA is releasably attached, by means of the method according to the invention. In this case, the fluid to be centrifuged is the elution buffer containing the DNA.

The centrifugation container is suitable for incubating the carrier member, to which the DNA is releasably attached, in the elution buffer. Accordingly, the container interior is designed for incubating the carrier member, to which the DNA is releasably attached, in the elution buffer. This means in particular that the elution buffer can flow through the outflow opening of the centrifugation container only under the effect of a centrifugal force and thus does not flow through the outflow opening during the incubation.

In addition, the centrifugation container is suitable for centrifuging. The centrifuging is used for separating the elution buffer containing the DNA from the carrier member. Accordingly, the centrifugation container is designed such that the elution buffer containing the DNA can flow through the outflow opening during the centrifuging while the carrier member is retained in the centrifugation container at a distance from the outflow opening by the retaining device. The retaining device is thus designed such that the carrier member cannot close the outflow opening in a covering or sealing manner.

According to one embodiment, the retaining device has at least one retaining element that extends to a certain extent into the container interior from an internal face of the container wall and/or an internal face of the container base. This allows the at least one retaining element and the centrifugation container to be formed in one piece and/or of the same material. For example, the at least one retaining element can be molded on the internal face of the container wall and/or on the internal face of the container base. The centrifugation container having the at least one retaining element can thus be produced particularly simply in one manufacturing step without having to assemble two or more components.

According to a further embodiment, the at least one retaining element is a rib, a knob, or a bar. The retaining element can thus have a basic shape that is particularly simple to produce. The rib can, for example, be of an octahedral shape having congruent trapezoidal side faces, or have a cuboidal basic shape. The octahedral shape having the congruent trapezoidal side faces is preferable in this case since it makes it simpler to eject the centrifugation container from the injection mold when the centrifugation container is produced using injection-molding technology.

As described above in relation to the method according to the invention, it is also possible that the retaining device does not have a retaining element. For example, the retaining device can be formed by the internal face of the container wall and/or the internal face of the container base, or the retaining device can be formed by the shape of the container interior of the centrifugation container. The embodiments of the retaining device that are described herein in relation to the method according to the invention also apply accordingly to the centrifugation container according to the invention.

According to one embodiment, the container base is formed conically in relation to a central longitudinal axis of the centrifugation container. As a result, the volume of the elution buffer can be kept low.

According to a further embodiment, the at least one retaining element extends to a certain extent into the container interior from the conical container base. In this case, the free end of the at least one retaining element can have the same incline as the conical container base in relation to the central longitudinal axis of the centrifugation container.

According to a further embodiment, a plurality of retaining elements are arranged in the region of the conical container base so as to extend radially outward in a radiating manner from the central longitudinal axis when viewed in a direction along the central longitudinal axis.

According to a further embodiment, a plurality of retaining elements are arranged in the container interior so as to extend radially outward in a radiating manner from the central longitudinal axis when viewed in a direction along the central longitudinal axis.

According to a further embodiment, the container wall is formed so as to be elongated beyond the container base in an axial direction along the central longitudinal axis, thus forming a supporting collar. The supporting collar allows the centrifugation container to be plugged on the collection vessel in a simple manner. In addition, the supporting collar makes handling the centrifugation container simpler. For example, the supporting collar can make it simpler to arrange the centrifugation container in its filling position and to stabilize the centrifugation container in its filling position.

According to a further embodiment, the supporting collar comprises a supporting shoulder, which is configured and designed to support the centrifugation container on a vessel rim of the collection vessel. The centrifugation container can thus be plugged on the collection vessel particularly simply and particularly sturdily.

According to a further embodiment, the supporting collar protrudes outside the container interior beyond an outlet opening of the outflow opening when viewed in the axial direction. This reduces the risk of the outlet opening of the outflow opening being touched when the centrifugation container is being handled. As a result, the risk of any contamination of the fluid to be centrifuged can be reduced.

According to a further embodiment, a plurality of centrifugation containers are formed in one piece, butted side by side in a linear manner to form a centrifugation container chain. This makes it simpler to concurrently process a plurality of samples. The centrifugation container chain has in particular eight centrifugation containers.

According to a further embodiment, a plurality of centrifugation containers are formed in one piece, butted side by side in a two-dimensional manner, thus forming a centrifugation container field. This likewise simplifies concurrent processing of a plurality of samples. The centrifugation container field has 96 centrifugation containers, for example, which are butted side by side in 12 rows with 8 centrifugation containers per row.

According to a further embodiment, at least one handle piece is molded integrally on the centrifugation container, the centrifugation container chain, or the centrifugation container field. The handle piece simplifies the handling, in particular manual handling, of the centrifugation container, the centrifugation container chain, or the centrifugation container field.

The invention further relates to a centrifugation container set for separating HMW DNA and/or UHMW DNA from a carrier member, to which the DNA is releasably attached, wherein the centrifugation container set has at least one centrifugation container according to the invention and at least one collection vessel, wherein the at least one centrifugation container and the at least one collection vessel are geometrically coordinated with one another such that when the centrifugation container is in the plugged-on state, there is a free distance from an outlet opening of the outflow opening of the centrifugation container to a vessel base of the collection vessel in the direction of a main extension axis of the centrifugation container, wherein the free distance is selected such that possible threads of the DNA detach from the outlet opening.

The centrifugation container set is suitable in particular for separating HMW DNA and/or UHMW DNA from a carrier member, to which the DNA is releasably attached, by means of the method according to the invention. When the centrifugation container is in the plugged-on state, the centrifugation container set is suitable for centrifuging in order to separate the elution buffer containing the DNA from the carrier member. Accordingly, when the centrifugation container is in the plugged-on state, the centrifugation container set is configured and designed such that during the centrifuging the elution buffer containing the DNA can flow through the outflow opening into the vessel interior of the collection vessel while the carrier member is retained in the centrifugation container at a distance from the outflow opening by the retaining device.

According to one embodiment, to implement different free distances, the centrifugation container set comprises centrifugation containers that have supporting collars having different longitudinal extensions from one another in the axial direction. In this way, different free distances can be implemented particularly simply.

The invention further relates to an automatic laboratory machine designed for carrying out a method according to the invention using a centrifugation container set according to the invention.

The invention further relates to a use of a centrifugation container according to the invention, of a centrifugation container set according to the invention, or of an automatic laboratory machine according to the invention for separating HMW DNA and/or UHMW DNA from a carrier member, to which the DNA is releasably attached.

The underlying principle of the invention can be used not only for separating HMW DNA and/or UHMW DNA from a carrier member, to which the DNA is releasably attached, but also for separating other biological materials from a carrier member, to which the biological materials are releasably attached.

Therefore, what is disclosed is a method for separating biological material from a carrier member, to which the biological material is releasably attached, using a centrifugation container set having at least one centrifugation container and at least one collection vessel, wherein the centrifugation container is separably plugged on the collection vessel, wherein the centrifugation container has a container wall, which surrounds a container interior, and a container base having at least one outflow opening, wherein the outflow opening forms at least one flow path from the container interior of the centrifugation container to a vessel interior of the collection vessel for a fluid to be centrifuged, wherein the at least one centrifugation container and the at least one collection vessel are geometrically coordinated with one another such that there is a free distance from an outlet opening of the outflow opening of the centrifugation container to a vessel base of the collection vessel in the direction of a main extension axis of the centrifugation container, wherein the free distance is selected such that possible threads of the biological material detach from the outlet opening, comprising the steps of:
incubating the carrier member, to which the biological material is releasably attached, in an elution fluid in the container interior of the centrifugation container,
centrifuging the centrifugation container set in order to separate the elution fluid containing the biological material from the carrier member, wherein during the centrifuging the elution fluid containing the biological material flows through the outflow opening into the vessel interior of the collection vessel while the carrier member is retained in the centrifugation container at a distance from the outflow opening by a retaining device.

As used herein, separating the biological material from the carrier member refers to a spatial separation of the biological material from the carrier member.

Preferably, the biological material is selected from the group consisting of nucleic acids, peptides, proteins, cells, and mixtures thereof. The nucleic acid can be DNA or RNA.

The elution fluid is a fluid by which the biological material can be separated from the carrier member during the centrifuging. The type or composition of the elution fluid is selected depending on the type of biological material. The elution fluid can be an elution buffer. By way of example, the elution buffer can be water or a slightly alkaline Tris-HCl solution.

At the beginning of the method, the biological material is releasably attached to the carrier member. The attachment to the carrier member is based on a non-covalent bond between the biological material and the carrier member, in which the biological material adheres to the surface of the carrier member. For this purpose, the carrier member has a biological material-binding (attaching) surface and shape that favors a gentle attachment of the biological material. The attachment of the biological material to the surface of the carrier member can be brought about by, among other things, electrostatic interactions and salt bridges between the biological material and the surface of the carrier member.

The carrier member can have numerous different shapes. All that is crucial is that, as described above, it has a surface to which the biological material can releasably attach.

Between the outlet opening of the outflow opening of the centrifugation container and the vessel base of the collection vessel there is the free distance in the direction of the main extension axis of the centrifugation container, wherein the free distance is selected such that possible threads of the biological material detach from the outlet opening. Threads of the biological material may appear when the incubation of the carrier member, to which the biological material is releasably attached, in the elution fluid leads to a viscous solution. Viscous solutions have the property of pulling threads. The threads appear particularly between the outlet opening of the outflow opening of the centrifugation container and the vessel base of the collection vessel when the centrifugation container set is centrifuged. The free distance between the outlet opening of the centrifugation container and the vessel base of the collection vessel therefore has to be sufficiently large so that such threads tear. To establish which minimum free distance should be selected so that possible threads of the biological material reliably detach from the outlet opening during the centrifuging, tests can be carried out using centrifugation container sets having different free distances.

The free distance is preferably at least 2.0 mm, more preferably at least 4.0 mm, more preferably at least 6.0 mm, more preferably at least 8.0 mm, more preferably at least 9.5 mm, more preferably at least 15.0 mm, more preferably at least 20.0 mm, more preferably at least 25.0 mm.

The features, embodiments, and advantages described in relation to the method according to the invention also apply accordingly to the method according to the disclosure.

Also disclosed is a centrifugation container for plugging on a collection vessel for separating biological material from a carrier member, to which the biological material is releasably attached, wherein the centrifugation container has:
a container wall, which surrounds a container interior, and
a container base having at least one outflow opening, wherein the outflow opening forms at least one flow path from the container interior to an external environment of the centrifugation container for a fluid to be centrifuged, wherein the container interior is designed for incubating the carrier member, to which the biological material is releasably attached, in an elution fluid, and wherein the centrifugation container has a retaining device that is designed for retaining the carrier member at a distance from the outflow opening.

The centrifugation container is suitable in particular for separating biological material from a carrier member, to which the biological material is releasably attached, by means of the method according to the disclosure. In this case, the fluid to be centrifuged is the elution fluid containing the biological material.

The features, embodiments, and advantages described in relation to the centrifugation container according to the invention also apply accordingly to the centrifugation container according to the disclosure.

Also disclosed is a centrifugation container set for separating biological material from a carrier member, to which the biological material is releasably attached, wherein the centrifugation container set has at least one centrifugation container according to the disclosure and at least one collection vessel, wherein the at least one centrifugation container and the at least one collection vessel are geometrically coordinated with one another such that when the centrifugation container is in the plugged-on state, there is a free distance from an outlet opening of the outflow opening of the centrifugation container to a vessel base of the collection vessel in the direction of a main extension axis of the centrifugation container, wherein the free distance is selected such that possible threads of the biological material detach from the outlet opening.

The centrifugation container set is suitable in particular for separating biological material from a carrier member, to which the biological material is releasably attached, by means of the method according to the disclosure.

The features, embodiments, and advantages described in relation to the centrifugation container set according to the invention also apply accordingly to the centrifugation container set according to the disclosure.

Also disclosed is an automatic laboratory machine designed for carrying out a method according to the disclosure using a centrifugation container set according to the disclosure.

Also disclosed is a use of a centrifugation container according to the disclosure, of a centrifugation container set according to the disclosure, or of an automatic laboratory machine according to the disclosure for separating biological material from a carrier member, to which the biological material is releasably attached.

The invention will now be explained with reference to the drawings, in which:

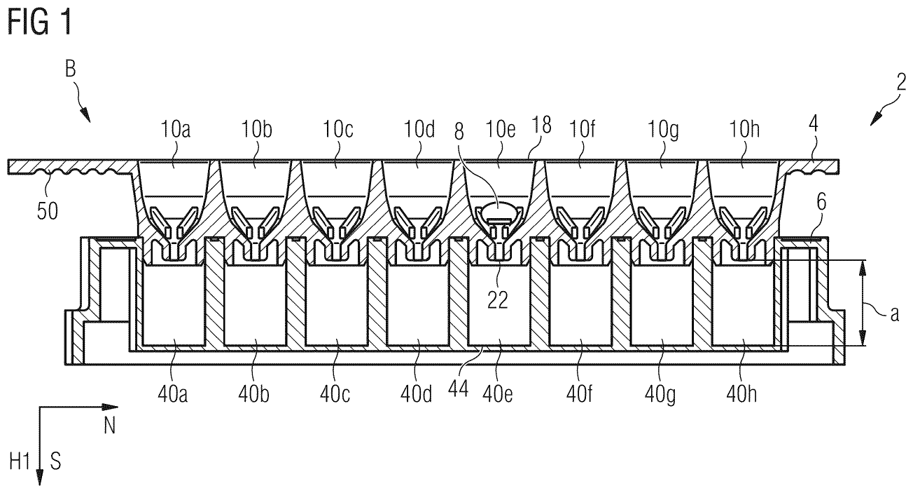

FIG. 1 is a schematic sectional view of components of a centrifugation container set for separating HMW DNA and/or UHMW DNA from a carrier member, to which the DNA is releasably attached.

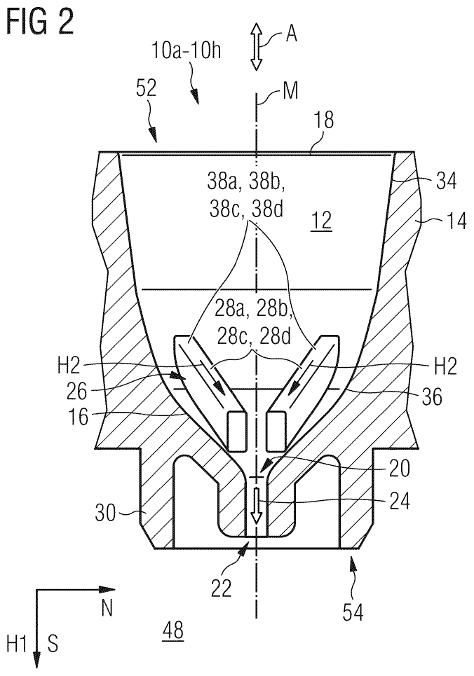

FIG. 2 is an enlarged schematic sectional view of a portion of FIG. 1.

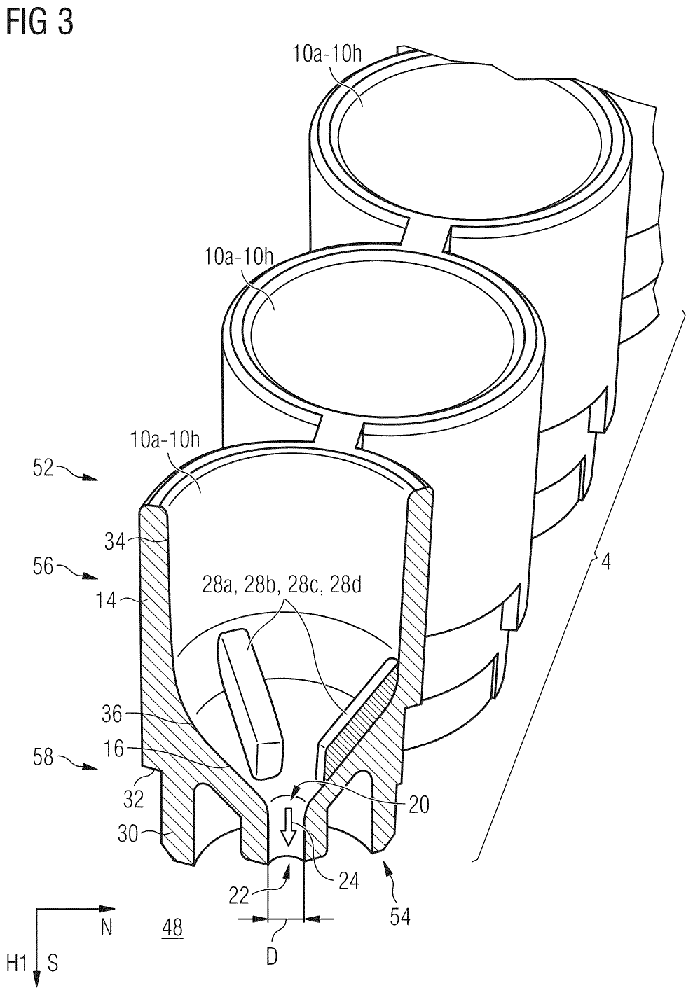

FIG. 3 is a schematic perspective view of a centrifugation container chain, the front centrifugation container being shown in a cut-open view.

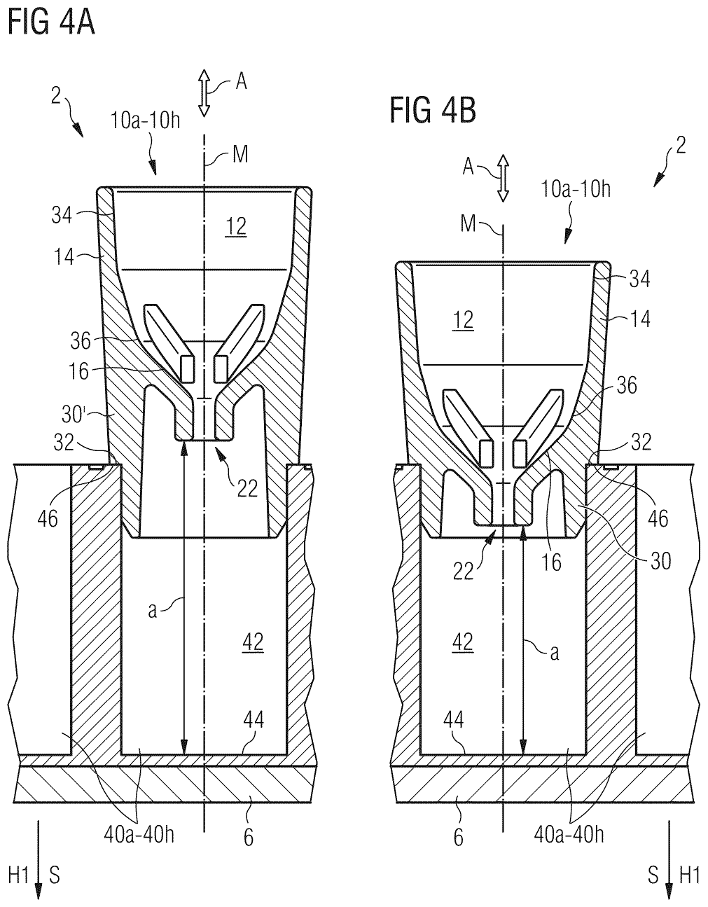

FIG. 4 is a schematic sectional view of components of a centrifugation container set comprising two different embodiment examples of a centrifugation container, with one centrifugation container comprising a longer supporting collar (FIG. 4A) and the other centrifugation container comprising a shorter supporting collar (FIG. 4B) in order to implement different free distances between the outflow opening of the centrifugation container and the vessel base of the collection vessel.

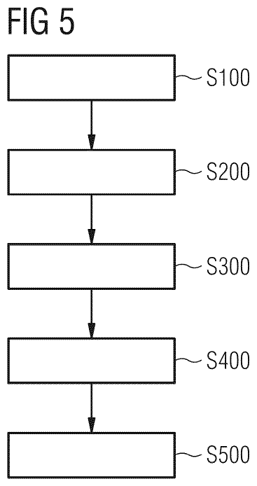

FIG. 5 is a schematic illustration of a method sequence for separating HMW DNA and/or UHMW DNA from a carrier member, to which the DNA is releasably attached.

Reference is first made to FIG. 1.

The figure shows a centrifugation container set 2 designed for separating HMW DNA and/or UHMW DNA from a carrier member 8, to which the DNA is releasably attached. In the present embodiment example, the carrier member 8 is a disk.

The centrifugation container set 2 comprises a centrifugation container chain 4 and a collection vessel chain 6.

In the present embodiment example, the centrifugation container chain 4 is a plastics injection-molded part formed in one piece and of the same material and comprises, in the present embodiment example, eight centrifugation containers 10a, 10b, 10c, 10d, 10e, 10f, 10g, 10h arranged in a row. The centrifugation containers 10a, 10b, 10c, 10d, 10e, 10f, 10g, 10h are butted side by side in a linear manner. Departing from the present embodiment example, the number of centrifugation containers 10a, 10b, 10c, 10d, 10e, 10f, 10g, 10h can also be different, for example one. Departing from the present embodiment example, a plurality of centrifugation containers 10a, 10b, 10c, 10d, 10e, 10f, 10g, 10h can be butted side by side in a two-dimensional manner, that is to say, in a plurality of rows and columns. In this way, a centrifugation container field formed in one piece can be formed. In this case, the centrifugation containers 10a, 10b, 10c, 10d, 10e, 10f, 10g, 10h are preferably arranged in the form of a microtiter plate. For example, the centrifugation container field can comprise 96 centrifugation containers 10a, 10b, 10c, 10d, 10e, 10f, 10g, 10h in the form of a 96-well microtiter plate. A 96-well microtiter plate is a microtiter plate comprising 96 isolated containers (wells), which can also be called cells and are arranged in 12 rows with 8 containers per row.

In the present embodiment example, the collection vessel chain 6 is also a plastics injection-molded part formed in one piece and of the same material and comprises, in the present embodiment example, eight collection vessels 40a, 40b, 40c, 40d, 40e, 40f, 40g, 40h arranged in a row. The collection vessels 40a, 40b, 40c, 40d, 40e, 40f, 40g, 40h are butted side by side in a linear manner. Departing from the present embodiment example, the number of collection vessels 40a, 40b, 40c, 40d, 40e, 40f, 40g, 40h can also be different, for example one. Departing from the present embodiment example, a plurality of collection vessels 40a, 40b, 40c, 40d, 40e, 40f, 40g, 40h can be butted side by side in a two-dimensional manner, that is to say, in a plurality of rows and columns. In this way, a collection vessel field formed in one piece can be formed. In this case, the collection vessels 40a, 40b, 40c, 40d, 40e, 40f, 40g, 40h are preferably arranged in the form of a microtiter plate. The collection vessel field can be a standard commercial microtiter plate, for example a 96-well microtiter plate having 96 collection vessels 40a, 40b, 40c, 40d, 40e, 40f, 40g, 40h. In this case, the collection vessels 40a, 40b, 40c, 40d, 40e, 40f, 40g, 40h are arranged in 12 rows with 8 collection vessels 40a, 40b, 40c, 40d, 40e, 40f, 40g, 40h per row.

The collection vessel 40a, 40b, 40c, 40d, 40e, 40f, 40g, 40h can also be called a collection container. Accordingly, the centrifugation container set 2 can also be called a container assembly, the centrifugation container chain 4 can be called a centrifugation container subassembly, and the collection vessel chain 6 can be called a collection container subassembly.

The centrifugation container chain 4 and the collection vessel chain 6 of the centrifugation container set 2 can be interconnected by a frictional fit without the use of tools and can likewise be separated again without the use of tools, as will be explained in detail below.

In the present embodiment example, the centrifugation container chain 4 comprises a handle piece 50 in the form of a holding extension, by which the handling of the centrifugation container chain 4 is simplified.

The centrifugation containers 10a, 10b, 10c, 10d, 10e, 10f, 10g, 10h of the centrifugation container chain 4 each have a container wall 14 (see FIG. 2) which surrounds a container interior 12 (see FIG. 2). In addition, the centrifugation containers 10a, 10b, 10c, 10d, 10e, 10f, 10g, 10h each have a container base 16 (see FIG. 2) having an outflow opening 20 (see FIG. 2).

In the present embodiment example, the container base 16 of each centrifugation container 10a, 10b, 10c, 10d, 10e, 10f, 10g, 10h is formed conically in relation to a central longitudinal axis M (see FIG. 2) of the centrifugation container 10a, 10b, 10c, 10d, 10e, 10f, 10g, 10h. In this case, the container base 16 tapers conically toward the outflow opening 20. Owing to the conical shape of the container base, the container interior 12 of the centrifugation container 10a, 10b, 10c, 10d, 10e, 10f, 10g, 10h is widened.

Thus, in the present embodiment example, the container interior 12 of each centrifugation container 10a, 10b, 10c, 10d, 10e, 10f, 10g, 10h in the filling position B shown in FIG. 1 has a tubular first portion 56, which is at the top in the direction of gravity S (see FIG. 3), and a second portion 58, which is at the bottom in the direction of gravity S and tapers conically toward the outflow opening 20 (see FIG. 3). The tubular portion 56 is surrounded by the container wall 14, and the portion 58 tapering conically toward the outflow opening 20 is surrounded by the container base 16. The tubular top portion 56 tapers slightly conically toward the bottom portion 58. This simplifies the production of the centrifugation container 10a, 10b, 10c, 10d, 10e, 10f, 10g, 10h.

The centrifugation containers 10a, 10b, 10c, 10d, 10e, 10f, 10g, 10h of the centrifugation container chain 4 each comprise a filling opening 18 through which the centrifugation containers 10a, 10b, 10c, 10d, 10e, 10f, 10g, 10h, or more precisely the container interior 12 thereof, can each be filled with a carrier member 8, to which the HMW DNA and/or UHMW DNA is releasably attached, and elution buffer. In FIG. 1, the carrier member 8 is shown in one of the centrifugation containers 10a, 10b, 10c, 10d, 10e, 10f, 10g, 10h by way of example.

Departing from the present embodiment example, the centrifugation container 10a, 10b, 10c, 10d, 10e, 10f, 10g, 10h can also take a different shape. For example, the top portion 56 can have a square cross section. Owing to the square cross section, the filling opening 18 and the container interior 12 can be configured to be larger. This makes it simpler to fill the centrifugation container 10a, 10b, 10c, 10d, 10e, 10f, 10g, 10h with the carrier member 8 and the elution buffer, thereby simplifying in particular automated filling with the carrier member 8.

The centrifugation container 10a, 10b, 10c, 10d, 10e, 10f, 10g, 10h can have a molded closure element having a cover that closes the filling opening 18, and a manually deformable connecting element that connects the cover to the centrifugation container 10a, 10b, 10c, 10d, 10e, 10f, 10g, 10h. The centrifugation container 10a, 10b, 10c, 10d, 10e, 10f, 10g, 10h and the closure element can be formed in one piece and of the same material. The centrifugation container chain 4 can also comprise a corresponding closure element.

In the filling position B shown in FIG. 1, the filling opening 18 of each centrifugation container 10a, 10b, 10c, 10d, 10e, 10f, 10g, 10h is in each case located at the top end 52 (see FIG. 2) of the centrifugation container 10a, 10b, 10c, 10d, 10e, 10f, 10g, 10h in the direction of gravity S.

As already mentioned, the centrifugation containers 10a, 10b, 10c, 10d, 10e, 10f, 10g, 10h of the centrifugation container chain 4 each have the container base 16 having the outflow opening 20. The outflow opening 20 forms a flow path 24 (see FIG. 2) for the fluid to be centrifuged from the container interior 12 of the centrifugation container 10a, 10b, 10c, 10d, 10e, 10f, 10g, 10h to a vessel interior 42 (see FIG. 4) of the collection vessel 40a, 40b, 40c, 40d, 40e, 40f, 40g, 40h. The outflow opening 20 does not have a filter element. During the centrifuging, the elution buffer containing the DNA can pass through the outflow opening 20 into the collection vessels 40a, 40b, 40c, 40d, 40e, 40f, 40g, 40h, or more precisely into the vessel interior 42 thereof. During the centrifuging, the centrifugation container chain 4 and the collection vessel chain 6 of the centrifugation container set 2 are interconnected, as shown in FIG. 1, that is to say, the centrifugation container chain 4 is plugged on the collection vessel chain 6 such that each centrifugation container 10a, 10b, 10c, 10d, 10e, 10f, 10g, 10h is plugged on a collection vessel 40a, 40b, 40c, 40d, 40e, 40f, 40g, 40h. During the centrifuging, the elution buffer containing the DNA can in each case flow through the outflow opening 20 into the vessel interior 42 of the corresponding collection vessel 40a, 40b, 40c, 40d, 40e, 40f, 40g, 40h.

Thus, each centrifugation container 10a, 10b, 10c, 10d, 10e, 10f, 10g, 10h in the present embodiment example has two openings, namely the filling opening 18 and the outflow opening 20. Between the two openings is the container interior 12 having a capacity of, for example, 100 μl to 2,200 μl. In the present embodiment example, the container interior 12 has a capacity of 250 μl. In the filling position B, the outflow opening 20 is arranged at the bottom end of the container interior 12 in the direction of gravity S.

The outflow opening 20 has an outlet opening 22. In the present embodiment example, in the filling position B the outlet opening 22 of each centrifugation container 10a, 10b, 10c, 10d, 10e, 10f, 10g, 10h of the centrifugation container chain 4 is in each case arranged close to the bottom end 54 (see FIG. 2) of the centrifugation container 10a, 10b, 10c, 10d, 10e, 10f, 10g, 10h in the direction of gravity S.

The centrifugation container 10a, 10b, 10c, 10d, 10e, 10f, 10g, 10h is suitable for incubating the carrier member 8, to which the HMW DNA and/or UHMW DNA is releasably attached, in the elution buffer. Accordingly, the container interior 12 is designed for incubating the carrier member 8, to which the DNA is releasably attached, in the elution buffer. This means that the container interior 12 can be filled with the carrier member 8, to which the DNA is releasably attached, and the elution buffer, and in particular that the elution buffer can flow through the outflow opening 20 of the centrifugation container 10a, 10b, 10c, 10d, 10e, 10f, 10g, 10h only under the effect of a centrifugal force and thus does not flow through the outflow opening 20 during the incubation.

The centrifugation container 10a, 10b, 10c, 10d, 10e, 10f, 10g, 10h is suitable for centrifuging. The centrifuging is used for separating the elution buffer containing the DNA from the carrier member 8. Accordingly, the centrifugation container 10a, 10b, 10c, 10d, 10e, 10f, 10g, 10h is formed such that the elution buffer containing the DNA can flow through the outflow opening 20 under the effect of a centrifugal force while the carrier member 8 is retained in the centrifugation container 10a, 10b, 10c, 10d, 10e, 10f, 10g, 10h at a distance from the outflow opening 20 by a retaining device 26, as will be explained in detail below.

When the centrifugation container 10a, 10b, 10c, 10d, 10e, 10f, 10g, 10h is in the plugged-on state, the centrifugation container set 2 is likewise suitable for centrifuging, wherein during the centrifuging the elution buffer containing the DNA can flow through the outflow opening 20 into the vessel interior 42 of the collection vessel 40a, 40b, 40c, 40d, 40e, 40f, 40g, 40h while the carrier member 8 is retained in the centrifugation container 10a, 10b, 10c, 10d, 10e, 10f, 10g, 10h at a distance from the outflow opening 20 by the retaining device 26.

In the present embodiment example, the carrier member 8 is a disk. The disk has a rotationally symmetrical, discoid basic shape. The surface of the disk is designed to bind HMW DNA and UHMW DNA. Suitable disks for binding HMW DNA and UHMW DNA are known. In the present embodiment example, the surface of the disk is covered with micro- and nanostructured silica.

The diameter of the disk can be in a range from 3.0 mm to 6.0 mm, for example. In the present embodiment example, the diameter of the disk is 4.0 mm.

In the present embodiment example, each collection vessel 40a, 40b, 40c, 40d, 40e, 40f, 40g, 40h comprises a tubular vessel interior 42, which comprises an inlet opening at the top in the direction of gravity S in the filling position B.

Departing from the present embodiment example, the collection vessel 40a, 40b, 40c, 40d, 40e, 40f, 40g, 40h can also have a different shape. For example, the collection vessel can be a standard reaction vessel having a substantially cup-shaped design and a capacity of 1.5 ml.

Furthermore, each collection vessel 40a, 40b, 40c, 40d, 40e, 40f, 40g, 40h comprises a vessel base 44, which is arranged at the bottom in the direction of gravity S in the filling position B. The centrifugation container 10a, 10b, 10c, 10d, 10e, 10f, 10g, 10h and the collection vessel 40a, 40b, 40c, 40d, 40e, 40f, 40g, 40h are geometrically coordinated with one another such that there is a free distance a from the outlet opening 22 of the outflow opening 20 of the centrifugation container 10a, 10b, 10c, 10d, 10e, 10f, 10g, 10h to the vessel base 44 of the collection vessel 40a, 40b, 40c, 40d, 40e, 40f, 40g, 40h in the direction of a main extension axis H1 of the centrifugation container 10a, 10b, 10c, 10d, 10e, 10f, 10g, 10h, wherein the free distance a is selected such that possible threads of the DNA detach from the outlet opening 22. The free distance a can also be called an air clearance. The free distance a ensures that possible threads of the DNA that may form during the centrifuging and may extend from the outlet opening 22 of the outflow opening 20 to the vessel base 44 detach from the outlet opening 22 while the centrifugation container set is being centrifuged and collect on the vessel base 44. Only in this way can the elution buffer containing the DNA be reliably collected in full in the collection vessel 40a, 40b, 40c, 40d, 40e, 40f, 40g, 40h. The threads are the result of the high viscosity of long DNA molecules. The appearance of possible threads of the DNA during the centrifuging depends on the viscosity of the DNA-containing elution buffer. Besides the length of the DNA molecules, the viscosity of the DNA-containing elution buffer also depends on the concentration of the accordingly long DNA molecules in the elution buffer. As the viscosity of the DNA-containing elution buffer increases, the free distance a from the outlet opening 22 of the centrifugation container 10a, 10b, 10c, 10d, 10e, 10f, 10g, 10h to the vessel base 44 of the collection vessel 40a, 40b, 40c, 40d, 40e, 40f, 40g, 40h must be increased accordingly.

The free distance a is preferably at least 6.0 mm, more preferably at least 8.0 mm, more preferably at least 9.5 mm. In the present embodiment example, a free distance a of 9.5 mm was selected.

Reference is now additionally made to FIGS. 2 and 3.

The main extension axis H1 of the centrifugation container 10a, 10b, 10c, 10d, 10e, 10f, 10g, 10h is the axis of the centrifugation container 10a, 10b, 10c, 10d, 10e, 10f, 10g, 10h in its largest extension. When the centrifugation container 10a, 10b, 10c, 10d, 10e, 10f, 10g, 10h is in the filling position B, said main extension axis H1 extends from the top end 52 of said centrifugation container to the bottom end 54 thereof, the direction of the main extension axis H1 coinciding with the direction of gravity S in the filling position B.

A secondary extension axis N of the centrifugation container 10a, 10b, 10c, 10d, 10e, 10f, 10g, 10h, on the other hand, extends radially inward or radially outward owing to the rotationally symmetrical configuration of the centrifugation container 10a, 10b, 10c, 10d, 10e, 10f, 10g, 10h.

The outflow opening 20 is dimensioned such that during the centrifuging the elution buffer containing the DNA reaches the collection vessel 40a, 40b, 40c, 40d, 40e, 40f, 40g, 40h through the outflow opening 20, and before the centrifuging the elution buffer is retained in the centrifugation container 10a, 10b, 10c, 10d, 10e, 10f, 10g, 10h in the filling position B by capillary forces and/or surface tension. For this purpose, the outflow opening 20 has a diameter D (see FIG. 3) in a range from preferably 0.6 mm to 1.6 mm, more preferably from 0.6 mm to 1.0 mm. In the present embodiment example, the outflow opening 20 has a diameter D of 1.0 mm. The length of the outflow opening 20 from the container base 16 to the outlet opening 22 is 2.0 mm in the present embodiment example.

To make it simpler to connect the centrifugation container 10a, 10b, 10c, 10d, 10e, 10f, 10g, 10h to the collection vessel 40a, 40b, 40c, 40d, 40e, 40f, 40g, 40h, a supporting collar 30, 30′ is provided in the present embodiment example (see FIGS. 2 to 4). The supporting collar 30, 30′ is formed by the container wall 14, the container wall 14 for this purpose being formed so as to be elongated beyond the container base 16 in an axial direction A along the central longitudinal axis M. In the filling position B, the supporting collar 30, 30′ comprises a portion that is arranged at the bottom end in the direction of gravity S and which can be engaged with a top portion of the collection vessel 40a, 40b, 40c, 40d, 40e, 40f, 40g, 40h in order to plug the centrifugation container 10a, 10b, 10c, 10d, 10e, 10f, 10g, 10h on the collection vessel 40a, 40b, 40c, 40d, 40e, 40f, 40g, 40h. In the present embodiment example, the supporting collar 30, 30′ is annular.

In the present embodiment example, the supporting collar 30, 30′ protrudes outside the container interior 12 beyond the outlet opening 22 of the outflow opening 20 when viewed in the axial direction A. The supporting collar 30, 30′ thus simplifies the handling of the centrifugation container 10a, 10b, 10c, 10d, 10e, 10f, 10g, 10h when it is not in the plugged-on state. The supporting collar 30, 30′ makes it simpler to arrange the centrifugation container 10a, 10b, 10c, 10d, 10e, 10f, 10g, 10h in its filling position B and stabilizes the centrifugation container 10a, 10b, 10c, 10d, 10e, 10f, 10g, 10h in its filling position B when it is not in the plugged-on state. Furthermore, since the supporting collar 30, 30′ protrudes outside the container interior 12 beyond the outlet opening 22 of the outflow opening 20 when viewed in the axial direction A, this reduces the risk of the outlet opening 22 of the outflow opening 20 being touched when the centrifugation container 10a, 10b, 10c, 10d, 10e, 10f, 10g, 10h is being handled. As a result, the risk of any contamination of the fluid to be centrifuged can be reduced.

In the present embodiment example, the supporting collar 30, 30′ comprises a supporting shoulder 32, which is configured and designed for supporting the centrifugation container 10a, 10b, 10c, 10d, 10e, 10f, 10g, 10h on a vessel rim 46 (see FIG. 4) of the collection vessel 40a, 40b, 40c, 40d, 40e, 40f, 40g, 40h. The vessel rim 46 of the collection vessel 40a, 40b, 40c, 40d, 40e, 40f, 40g, 40h is located at the top end of the collection vessel 40a, 40b, 40c, 40d, 40e, 40f, 40g, 40h in the direction of gravity S in the filling position B. Owing to the supporting shoulder 32, the centrifugation container 10a, 10b, 10c, 10d, 10e, 10f, 10g, 10h can be plugged on the collection vessel 40a, 40b, 40c, 40d, 40e, 40f, 40g, 40h particularly simply and particularly sturdily.

Furthermore, in the present embodiment example the centrifugation container 10a, 10b, 10c, 10d, 10e, 10f, 10g, 10h has the retaining device 26, which is arranged in the container interior 12 and is designed for retaining the carrier member 8 at a distance from the outflow opening 20. The retaining device 26 is thus designed such that the carrier member 8 cannot close the outflow opening 20 in a covering or sealing manner. For this purpose, the retaining device 26 in the present embodiment example has four retaining elements 28a, 28b, 28c, 28d. Departing from the present embodiment example, the number of retaining elements 28a, 28b, 28c, 28d can also be different, for example one. In the present embodiment example, the retaining elements 28a, 28b, 28c, 28d extend to a certain extent into the container interior 12 from an internal face 36 of the container base 16. More precisely, the retaining elements 28a, 28b, 28c, 28d extend to a certain extent into the container interior 12 from the internal face 36 of the conical container base 16. Thus, the retaining elements 28a, 28b, 28c, 28d are arranged in the bottom portion 58 of the centrifugation container 10a, 10b, 10c, 10d, 10e, 10f, 10g, 10h, which bottom portion tapers conically toward the outflow opening 20. In this case, the free ends of the retaining elements 28a, 28b, 28c, 28d have the same incline as the conical container base 16 in relation to the central longitudinal axis M of the centrifugation container 10a, 10b, 10c, 10d, 10e, 10f, 10g, 10h. Furthermore, in the present embodiment example the retaining elements 28a, 28b, 28c, 28d are arranged in the region of the conical container base 16 so as to extend radially outward in a radiating manner from the central longitudinal axis M when viewed in a direction along the central longitudinal axis M.

Departing from the present embodiment example, the retaining elements 28a, 28b, 28c, 28d can extend to a certain extent into the container interior 12 from an internal face 34 of the container wall 14. The retaining elements 28a, 28b, 28c, 28d can furthermore extend to a certain extent into the container interior 12 from both the internal face 34 of the container wall 14 and the internal face 36 of the container base 16. In both cases, the retaining elements 28a, 28b, 28c, 28d can, for example, be arranged in the container interior 12 so as to extend radially outward in a radiating manner from the central longitudinal axis M when viewed in a direction along the central longitudinal axis M.

Each retaining element 28a, 28b, 28c, 28d has the shape of a rib in the present embodiment example. In this case, each retaining element 28a, 28b, 28c, 28d is formed substantially cuboidally. The cuboidal ribs have a width of approximately 1.0 mm and a height of approximately 1.0 mm in the present embodiment example. Departing from the present embodiment example, the rib can also take the shape of an octahedron having congruent trapezoidal side faces. Departing from the present embodiment example, the shape of the retaining element 28a, 28b, 28c, 28d can also be different, for example a knob or a bar.

Where there are a plurality of retaining elements 28a, 28b, 28c, 28d, the retaining elements 28a, 28b, 28c, 28d can take different shapes from one another, departing from the present embodiment example.

The centrifugation container 10a, 10b, 10c, 10d, 10e, 10f, 10g, 10h and the retaining elements 28a, 28b, 28c, 28d are formed in one piece and of the same material in the present embodiment example.

The retaining elements 28a, 28b, 28c, 28d each provide at least one contact surface 38a, 38b, 38c, 38d for coming into contact with a portion of the carrier member 8. For this purpose, in the present embodiment example the contact surface 38a, 38b, 38c, 38d is arranged at a distance from the internal face 36 of the container base 16 inward into the container interior.

In the present embodiment example, the retaining elements 28a, 28b, 28c, 28d are arranged at a regular distance from one another owing to the rotationally symmetrical configuration of the centrifugation container 10a, 10b, 10c, 10d, 10e, 10f, 10g, 10h.

Owing to their arrangement in the tapering bottom portion 58, the retaining elements 28a, 28b, 28c, 28d each have a main extension axis H2 the direction of which is located between the main extension axis H1 and the secondary extension axis N. The direction of the main extension axis H2 can deviate from the directions of the main extension axis H1 or secondary extension axis N by an angle of 30° to 60°.

Owing to the retaining device 26, a carrier member 8 resting on a retaining element 28a, 28b, 28c, 28d cannot close the outflow opening 20; rather, at least one channel leading to the outflow opening 20 remains open. In the at least one channel, the fluid to be centrifuged can reach the outflow opening 20. The at least one channel can be formed between a plurality of retaining elements 28a, 28b, 28c, 28d. In the present embodiment example, there is one such channel between each adjacent retaining element 28a, 28b, 28c, 28d, such that four channels are formed between the retaining elements 28a, 28b, 28c, 28d when the carrier member 8 rests on the retaining elements 28a, 28b, 28c, 28d. In the present embodiment example, the channels are each 0.8 mm wide and 0.75 mm high. In the present embodiment example, the action of gravity is enough to ensure that the fluid to be centrifuged reaches the outflow opening 20. Departing from the present embodiment example, the fluid to be centrifuged can reach the outflow opening 20 in the at least one channel only under the effect of a centrifugal force. In this case, the at least one channel has an accordingly small channel diameter.

The distance obtained by the retaining device 26 between the carrier member 8 and the outflow opening 20 is preferably in a range from 1.0 mm to 5.0 mm. In the present embodiment example, the distance is 3.5 mm.

Reference is now additionally made to FIG. 4.

FIGS. 4A and 4B are each sectional views of a centrifugation container set 2 having a centrifugation container 10a, 10b, 10c, 10d, 10e, 10f, 10g, 10h which is plugged on a collection vessel 40a, 40b, 40c, 40d, 40e, 40f, 40g, 40h. In this case, the portion of the supporting collar 30, 30′ arranged at the bottom end in the direction of gravity S in the filling position B is engaged with the top portion of the collection vessel 40a, 40b, 40c, 40d, 40e, 40f, 40g, 40h, and the supporting shoulder 32 of the supporting collar 30, 30′ supports the centrifugation container 10a, 10b, 10c, 10d, 10e, 10f, 10g, 10h on the vessel rim 46 of the collection vessel 40a, 40b, 40c, 40d, 40e, 40f, 40g, 40h.

As shown, to implement different free distances a, the centrifugation container set 2 can have centrifugation containers 10a, 10b, 10c, 10d, 10e, 10f, 10g, 10h that have supporting collars 30, 30′ having different longitudinal extensions from one another in the axial direction A. The centrifugation container 10a, 10b, 10c, 10d, 10e, 10f, 10g, 10h shown in FIG. 4A has a supporting collar 30′ that has a greater longitudinal extension in the axial direction A than the supporting collar 30 of the centrifugation container 10a, 10b, 10c, 10d, 10e, 10f, 10g, 10h shown in FIG. 4B. In other words, the centrifugation container 10a, 10b, 10c, 10d, 10e, 10f, 10g, 10h shown in FIG. 4A has a longer supporting collar 30′, and the centrifugation container 10a, 10b, 10c, 10d, 10e, 10f, 10g, 10h shown in FIG. 4B has a shorter supporting collar 30. Different free distances a are thus obtained. In the embodiment example shown in FIG. 4A, the free distance a is 13.0 mm. In the embodiment example shown in FIG. 4B, the free distance a is 9.5 mm.

In both embodiment examples, the supporting collar 30, 30′ protrudes outside the container interior 12 beyond the outlet opening 22 of the outflow opening 20 when viewed in the axial direction A. In this case, the longer supporting collar 30′ shown in FIG. 4A protrudes beyond the outlet opening 22 to a greater extent than the supporting collar 30 shown in FIG. 4B.

Departing from the present embodiment example, to implement the free distance a, the centrifugation container set 2 can comprise a spacer formed as a separate component that is inserted in the centrifugation container 10a, 10b, 10c, 10d, 10e, 10f, 10g, 10h and/or the collection vessel 40a, 40b, 40c, 40d, 40e, 40f, 40g, 40h. Alternatively, the spacer can also be molded on the centrifugation container 10a, 10b, 10c, 10d, 10e, 10f, 10g, 10h or on the collection vessel 40a, 40b, 40c, 40d, 40e, 40f, 40g, 40h, that is to say, the spacer and the centrifugation container 10a, 10b, 10c, 10d, 10e, 10f, 10g, 10h or the spacer and the collection vessel 40a, 40b, 40c, 40d, 40e, 40f, 40g, 40h are formed in one piece. In addition, the spacer and the centrifugation container 10a, 10b, 10c, 10d, 10e, 10f, 10g, 10h or the spacer and the collection vessel 40a, 40b, 40c, 40d, 40e, 40f, 40g, 40h can be formed of the same material.

A method for operating the centrifugation container set 2 will now be explained with additional reference to FIG. 5.

For this purpose, the centrifugation container set 2 having the centrifugation container chain 4 and the collection vessel chain 6 is used. The method is carried out at a temperature of 20° C. to 23° C. (room temperature). However, the method can also be carried out at a lower or a higher temperature, for example at a temperature of 37° C.

In a first step S100, the centrifugation container chain 4 having the centrifugation containers 10a, 10b, 10c, 10d, 10e, 10f, 10g, 10h is plugged on the collection vessel chain 6 comprising the collection vessels 40a, 40b, 40c, 40d, 40e, 40f, 40g, 40h. In this way, the centrifugation container chain 4 and the collection vessel chain 6 are separably interconnected.

In a further step S200, the carrier member 8, to which the HMW DNA and/or UHMW DNA is releasably attached, and the elution buffer are introduced into the container interior 12 of the centrifugation container 10a, 10b, 10c, 10d, 10e, 10f, 10g, 10h in the filling position B through the filling opening 18. In this case, the carrier member 8 and the elution buffer are preferably introduced into the container interior 12 of the centrifugation container 10a, 10b, 10c, 10d, 10e, 10f, 10g, 10h one after the other. In this regard, the carrier member 8 can be introduced into the centrifugation container 10a, 10b, 10c, 10d, 10e, 10f, 10g, 10h first, followed by the elution buffer, or preferably the elution buffer is introduced first, followed by the carrier member 8. In the present embodiment example, the elution buffer is introduced into the centrifugation container 10a, 10b, 10c, 10d, 10e, 10f, 10g, 10h first, followed by the carrier member 8. In this case, the elution buffer is pipetted into the centrifugation container 10a, 10b, 10c, 10d, 10e, 10f, 10g, 10h in an automated manner, and then the carrier member 8 is transferred into the elution buffer in an automated manner.

The DNA releasably attached to the carrier member 8 in the present embodiment example is UHMW DNA that has been isolated from 1.0 million cells of a human leukemia cell line (HL-60).

The volume of elution buffer can be in a range from 50 μl to 250 μl, for example. The volume of elution buffer is selected such that the carrier member 8 is entirely covered with the elution buffer in the container interior 12 of the centrifugation container 10a, 10b, 10c, 10d, 10e, 10f, 10g, 10h. In the present embodiment example, 100 μl of elution buffer are used.

In a further step S300, the carrier member 8, to which the DNA is releasably attached, is incubated in the elution buffer in the container interior 12 of the centrifugation container 10a, 10b, 10c, 10d, 10e, 10f, 10g, 10h.

The elution buffer is a fluid by which the DNA can be separated from the carrier member 8 during the centrifuging. Suitable elution buffers for releasing DNA releasably attached to a surface are known. By way of example, the elution buffer can be water or a slightly alkaline Tris-HCl solution. A Tris-HCl solution is an aqueous solution of Tris (2-amino-2-(hydroxymethyl) propane-1,3-diol), in which the desired pH has been set by adding HCl (hydrochloric acid) to the Tris solution. The slightly alkaline Tris-HCl solution typically has a pH in the range from 8.0 to 8.5. The slightly alkaline Tris-HCl solution can comprise 10 mM Tris, for example. It can also comprise EDTA (ethylenediaminetetraacetic acid), for example in a concentration from 0.1 to 1.0 mM. In the present embodiment example, a Tris-HCl solution comprising 10 mM Tris and having a pH of 8.0 is used as the elution buffer.

The incubation is typically carried out for 10 to 30 min, preferably for approximately 20 to 30 min. A shorter or longer incubation duration, for example overnight incubation, can also be selected. The incubation duration merely has to be sufficiently long so that the DNA can be separated from the carrier member 8 during the subsequent centrifuging. In the present embodiment example, the incubation is carried out for 20 min.

In a further step S400, the interconnected centrifugation container chain 4 and collection vessel chain 6 are centrifuged in order to separate the elution buffer containing the DNA from the carrier member 8.

The centrifuging is typically carried out at at least 1,500×g or at least 2,000×g, for example at 2,200×g. The centrifuging is typically carried out for at least 30 seconds, for example for 30 to 60 seconds, in order to ensure the elution buffer together with the DNA contained therein is fully separated from the carrier member 8. In the present embodiment example, the centrifuging is carried out at 2,200×g for 30 seconds.

In the present embodiment example, the retaining device 26 retains the carrier member 8 at a distance from the outflow opening 20 using its retaining elements 28a, 28b, 28c, 28d so that the outflow opening 20 is not closed by the carrier member 8.

Furthermore, during the centrifuging the capillary forces or surface tension of the elution buffer are not sufficient to prevent the elution buffer from exiting through the outflow opening 20.

During the centrifuging, the elution buffer together with the DNA contained therein flows through the outflow opening 20 of the centrifugation container 10a, 10b, 10c, 10d, 10e, 10f, 10g, 10h into the vessel interior 42 of the collection vessel 40a, 40b, 40c, 40d, 40e, 40f, 40g, 40h such that the elution buffer containing the DNA collects in the collection vessel 40a, 40b, 40c, 40d, 40e, 40f, 40g, 40h. In this case, the total volume of the elution buffer together with the DNA contained therein is transferred into the collection vessel 40a, 40b, 40c, 40d, 40e, 40f, 40g, 40h in one step. By contrast, the carrier member 8 remains in the centrifugation container 10a, 10b, 10c, 10d, 10e, 10f, 10g, 10h during the centrifuging. Thus, the centrifuging causes the DNA to spatially separate from the carrier member 8 to which the DNA was releasably attached. The DNA separated from the carrier member 8 is thus in the collection vessel 40a, 40b, 40c, 40d, 40e, 40f, 40g, 40h ready to be analyzed further, used in a different way, or stored.

In a further step S500, the centrifugation container chain 4 comprising the centrifugation containers 10a, 10b, 10c, 10d, 10e, 10f, 10g, 10h is then separated from the collection vessel chain 6 comprising the collection vessels 40a, 40b, 40c, 40d, 40e, 40f, 40g, 40h, manually without the use of tools. Alternatively, it is possible to separate the centrifugation container chain 4 from the collection vessel 6 in a mechanized, in particular automated, manner. In this way, the elution buffer containing the DNA is spatially accessible in the collection vessel 40a, 40b, 40c, 40d, 40e, 40f, 40g, 40h.

Departing from the present embodiment example, the order of the steps can also be different. For example, steps S100 and S200 can be carried out in reverse order, or step S100 can be carried out after step S300. Furthermore, a plurality of steps can also be carried out at the same time or simultaneously. For example, steps S100 and S300 can be carried out at the same time or simultaneously. In addition, individual steps can be skipped or omitted, departing from the present embodiment example. For example, step S100 can be omitted if the centrifugation container chain 4 having the centrifugation containers 10a, 10b, 10c, 10d, 10e, 10f, 10g, 10h is already plugged on the collection vessel chain 6 comprising the collection vessels 40a, 40b, 40c, 40d, 40e, 40f, 40g, 40h.

Using the method, HMW DNA and/or UHMW DNA can be reliably separated from the carrier member 8, and the DNA can be reliably prevented from undesirably binding to the carrier member 8 again. In addition, the method can ensure that no DNA remains stuck to the carrier member 8.

Moreover, the method does not need the at least two pipetting steps that are required in the previously known procedure, and it can be carried out in an automated manner.

### LIST OF REFERENCE SIGNS

2 Centrifugation container set
4 Centrifugation container chain
6 Collection vessel chain
8 Carrier member
10a Centrifugation container
10b Centrifugation container
10c Centrifugation container
10d Centrifugation container
10e Centrifugation container
10f Centrifugation container
10g Centrifugation container
10h Centrifugation container
12 Container interior
14 Container wall
16 Container base
18 Filling opening
20 Outflow opening
22 Outlet opening
24 Flow path
26 Retaining device
28a Retaining element
28b Retaining element
28c Retaining element
28d Retaining element
30 Supporting collar
30′ Supporting collar
32 Supporting shoulder
34 Internal face of the container wall
36 Internal face of the container base
38a Contact surface
38b Contact surface
38c Contact surface
38d Contact surface
40a Collection vessel
40b Collection vessel
40c Collection vessel
40d Collection vessel
40e Collection vessel
40f Collection vessel
40g Collection vessel
40h Collection vessel
42 Vessel interior
44 Vessel base
46 Vessel rim
48 External environment
50 Handle piece
52 Top end
54 Bottom end
56 Top portion
58 Bottom portion
a Free distance
B Filling position
D Diameter
M Central longitudinal axis
H1 Main extension axis
H2 Main extension axis
N Secondary extension axis
A Axial direction
S Direction of gravity
S100 Step
S200 Step
S300 Step
S400 Step
S500 Step

1. A method for separating HMW DNA and/or UHMW DNA from a carrier member (8), to which the DNA is releasably attached, using a centrifugation container set (2) having at least one centrifugation container (10a, 10b, 10c, 10d, 10e, 10f, 10g, 10h) and at least one collection vessel (40a, 40b, 40c, 40d, 40e, 40f, 40g, 40h), wherein the centrifugation container (10a, 10b, 10c, 10d, 10e, 10f, 10g, 10h) is separably plugged on the collection vessel (40a, 40b, 40c, 40d, 40e, 40f, 40g, 40h), wherein the centrifugation container (10a, 10b, 10c, 10d, 10e, 10f, 10g, 10h) has a container wall (14), which surrounds a container interior (12), and a container base (16) having at least one outflow opening (20), wherein the outflow opening (20) forms at least one flow path (24) from the container interior (12) of the centrifugation container (10a, 10b, 10c, 10d, 10e, 10f, 10g, 10h) to a vessel interior (42) of the collection vessel (40a, 40b, 40c, 40d, 40e, 40f, 40g, 40h) for a fluid to be centrifuged, wherein the at least one centrifugation container (10a, 10b, 10c, 10d, 10e, 10f, 10g, 10h) and the at least one collection vessel (40a, 40b, 40c, 40d, 40e, 40f, 40g, 40h) are geometrically coordinated with one another such that there is a free distance (a) from an outlet opening (22) of the outflow opening (20) of the centrifugation container (10a, 10b, 10c, 10d, 10e, 10f, 10g, 10h) to a vessel base (44) of the collection vessel (40a, 40b, 40c, 40d, 40e, 40f, 40g, 40h) in the direction of a main extension axis (H1) of the centrifugation container (10a, 10b, 10c, 10d, 10e, 10f, 10g, 10h), wherein the free distance (a) is selected such that possible threads of the DNA detach from the outlet opening (22), comprising the steps of:
incubating the carrier member (8), to which the DNA is releasably attached, in an elution buffer in the container interior (12) of the centrifugation container (10a, 10b, 10c, 10d, 10e, 10f, 10g, 10h),
centrifuging the centrifugation container set (2) in order to separate the elution buffer containing the DNA from the carrier member (8), wherein during the centrifuging the elution buffer containing the DNA flows through the outflow opening (20) into the vessel interior (42) of the collection vessel (40a, 40b, 40c, 40d, 40e, 40f, 40g, 40h) while the carrier member (8) is retained in the centrifugation container (10a, 10b, 10c, 10d, 10e, 10f, 10g, 10h) at a distance from the outflow opening (20) by a retaining device (26).

2. The method according to claim 1, wherein the retaining device (26) has at least one retaining element (28a, 28b, 28c, 28d) that extends to a certain extent into the container interior (12) from an internal face (34) of the container wall (14) and/or an internal face (36) of the container base (16).

3. The method according to claim 1, wherein the container wall (14) and/or the container base (16) form the retaining device (26).

4. The method according to claim 1, wherein the DNA is UHMW DNA and the free distance (a) is at least 8.0 mm.

5. The method according to claim 1, wherein the carrier member is a disk having a diameter of 1.0 mm to 6.0 mm.

6. A centrifugation container (10a, 10b, 10c, 10d, 10e, 10f, 10g, 10h) for plugging on a collection vessel (40a, 40b, 40c, 40d, 40e, 40f, 40g, 40h) for separating HMW DNA and/or UHMW DNA from a carrier member (8), to which the DNA is releasably attached, wherein the centrifugation container (10a, 10b, 10c, 10d, 10e, 10f, 10g, 10h) has:
a container wall (14), which surrounds a container interior (12), and
a container base (16) having at least one outflow opening (20), wherein the outflow opening (20) forms at least one flow path (24) from the container interior (12) to an external environment (48) of the centrifugation container (10a, 10b, 10c, 10d, 10e, 10f, 10g, 10h) for a fluid to be centrifuged,
wherein the container interior (12) is designed for incubating the carrier member (8), to which the DNA is releasably attached, in an elution buffer, and
wherein the centrifugation container (10a, 10b, 10c, 10d, 10e, 10f, 10g, 10h) has a retaining device (26) that is designed for retaining the carrier member (8) at a distance from the outflow opening (20).

7. The centrifugation container (10a, 10b, 10c, 10d, 10e, 10f, 10g, 10h) according to claim 6, wherein the retaining device (26) has at least one retaining element (28a, 28b, 28c, 28d) that extends to a certain extent into the container interior (12) from an internal face (34) of the container wall (14) and/or an internal face (36) of the container base (16).

8. The centrifugation container (10a, 10b, 10c, 10d, 10e, 10f, 10g, 10h) according to claim 6, wherein the container wall (14) and/or the container base (16) form the retaining device (26).

9. The centrifugation container (10a, 10b, 10c, 10d, 10e, 10f, 10g, 10h) according to claim 6, wherein the container base (16) is formed conically in relation to a central longitudinal axis (M) of the centrifugation container (10a, 10b, 10c, 10d, 10e, 10f, 10g, 10h).

10. The centrifugation container (10a, 10b, 10c, 10d, 10e, 10f, 10g, 10h) according to claim 6, wherein the container wall (14) is formed so as to be elongated beyond the container base (16) in an axial direction (A) along the central longitudinal axis (M), thus forming a supporting collar (30, 30′).

11. The centrifugation container (10a, 10b, 10c, 10d, 10e, 10f, 10g, 10h) according to claim 10, wherein the supporting collar (30, 30′) protrudes outside the container interior (12) beyond an outlet opening (22) of the outflow opening (20) when viewed in the axial direction (A).

12. The centrifugation container (10a, 10b, 10c, 10d, 10e, 10f, 10g, 10h) according to claim 6, wherein a plurality of centrifugation containers (10a, 10b, 10c, 10d, 10e, 10f, 10g, 10h) are formed in one piece, butted side by side in a linear manner to form a centrifugation container chain (4).

13. The centrifugation container (10a, 10b, 10c, 10d, 10e, 10f, 10g, 10h) according to claim 6, wherein a plurality of centrifugation containers (10a, 10b, 10c, 10d, 10e, 10f, 10g, 10h) are formed in one piece, butted side by side in a two-dimensional manner, thus forming a centrifugation container field.

14. A centrifugation container set (2) for separating HMW DNA and/or UHMW DNA from a carrier member (8), to which the DNA is releasably attached, wherein the centrifugation container set (2) has at least one centrifugation container (10a, 10b, 10c, 10d, 10e, 10f, 10g, 10h) according to claim 6 and at least one collection vessel (40a, 40b, 40c, 40d, 40e, 40f, 40g, 40h), wherein the at least one centrifugation container (10a, 10b, 10c, 10d, 10e, 10f, 10g, 10h) and the at least one collection vessel (40a, 40b, 40c, 40d, 40e, 40f, 40g, 40h) are geometrically coordinated with one another such that when the centrifugation container (10a, 10b, 10c, 10d, 10e, 10f, 10g, 10h) is in the plugged-on state, there is a free distance (a) from an outlet opening (22) of the outflow opening (20) of the centrifugation container (10a, 10b, 10c, 10d, 10e, 10f, 10g, 10h) to a vessel base (44) of the collection vessel (40a, 40b, 40c, 40d, 40e, 40f, 40g, 40h) in the direction of a main extension axis (H1) of the centrifugation container (10a, 10b, 10c, 10d, 10e, 10f, 10g, 10h), wherein the free distance (a) is selected such that possible threads of the DNA detach from the outlet opening (22).

15. The centrifugation container set (2) according to claim 14, wherein, to implement different free distances (a), the centrifugation container set (2) has centrifugation containers (10a, 10b, 10c, 10d, 10e, 10f, 10g, 10h) that have supporting collars (30, 30′) having different longitudinal extensions from one another in the axial direction (A).

16. Automatic laboratory machine designed for carrying out a method according to claim 1 using a centrifugation container set (2) for separating HMW DNA and/or UHMW DNA from a carrier member (8), to which the DNA is releasably attached, wherein the centrifugation container set (2) has at least one centrifugation container (10a, 10b, 10c, 10d, 10e, 10f, 10g, 10h) and at least one collection vessel (40a, 40b, 40c, 40d, 40e, 40f, 40g, 40h), wherein the at least one centrifugation container (10a, 10b, 10c, 10d, 10e, 10f, 10g, 10h) is for plugging on the collection vessel (40a, 40b, 40c, 40d, 40e, 40f, 40g, 40h) for separating HMW DNA and/or UHMW DNA from a carrier member (8), to which the DNA is releasably attached, wherein the centrifugation container (10a, 10b, 10c, 10d, 10e, 10f, 10g, 10h) has:
a container wall (14), which surrounds a container interior (12), and
a container base (16) having at least one outflow opening (20), wherein the outflow opening (20) forms at least one flow path (24) from the container interior (12) to an external environment (48) of the centrifugation container (10a, 10b, 10c, 10d, 10e, 10f, 10g, 10h) for a fluid to be centrifuged,
wherein the container interior (12) is designed for incubating the carrier member (8), to which the DNA is releasably attached, in an elution buffer, and
wherein the centrifugation container (10a, 10b, 10c, 10d, 10e, 10f, 10g, 10h) has a retaining device (26) that is designed for retaining the carrier member (8) at a distance from the outflow opening (20), wherein the at least one centrifugation container (10a, 10b, 10c, 10d, 10e, 10f, 10g, 10h) and the at least one collection vessel (40a, 40b, 40c, 40d, 40e, 40f, 40g, 40h) are geometrically coordinated with one another such that when the centrifugation container (10a, 10b, 10c, 10d, 10e, 10f, 10g, 10h) is in the plugged-on state, there is a free distance (a) from an outlet opening (22) of the outflow opening (20) of the centrifugation container (10a, 10b, 10c, 10d, 10e, 10f, 10g, 10h) to a vessel base (44) of the collection vessel (40a, 40b, 40c, 40d, 40e, 40f, 40g, 40h) in the direction of a main extension axis (H1) of the centrifugation container (10a, 10b, 10c, 10d, 10e, 10f, 10g, 10h), wherein the free distance (a) is selected such that possible threads of the DNA detach from the outlet opening (22).

17. (canceled)
